(19) United States
(12) Patent Application Publication
(10) Pub. No.: US 2006/0289021 A1
(43) Pub. Date: Dec. 28, 2006

(54) ULTRAVIOLET THERAPIES FOR SPINE-RELATED PAIN

(76) Inventors: Thomas M. DiMauro, Southboro, MA (US); Mohamed Attawia, Canton, MA (US); Richard Toselli, Barrington, RI (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/468,930

(22) Filed: Aug. 31, 2006

Related U.S. Application Data
(62) Division of application No. 11/018,470, filed on Dec. 21, 2004.

(51) Int. Cl. A61B 18/18

(52) U.S. Cl. 128/898; 606/3; 606/14

(57) ABSTRACT

A UV probe capable of providing a Th2 immune environment for the treatment of back pain.

### CONTINUING DATA

This divisional patent application is a divisional of copending U.S. patent application Ser. No. 11/018,470, entitled “Ultraviolet Therapies for Spine-Related Pain”, filed Dec. 21, 2004, the specification of which is incorporated by reference in its entirety.

### BACKGROUND OF THE INVENTION

The natural intervertebral disc contains a jelly-like nucleus pulposus surrounded by a fibrous annulus fibrosus. Under an axial load, the nucleus pulposus compresses and radially transfers that load to the annulus fibrosus. The laminated nature of the annulus fibrosus provides it with a high tensile strength and so allows it to expand radially in response to this transferred load.

In a healthy intervertebral disc, cells within the nucleus pulposus produce an extracellular matrix (ECM) containing a high percentage of proteoglycans. These proteoglycans contain sulfated functional groups that retain water, thereby providing the nucleus pulposus within its cushioning qualities. These nucleus pulposus cells may also secrete small amounts of cytokines such as interleukin-1β and TNF-α as well as matrix metalloproteinases (“MMPs”). These cytokines and MMPs help regulate the metabolism of the nucleus pulposus cells.

In some instances of disc degeneration disease (DDD), gradual degeneration of the intervetebral disc is caused by mechanical instabilities in other portions of the spine. In these instances, increased loads and pressures on the nucleus pulposus cause the cells within the disc (or invading macrophases) to emit larger than normal amounts of the above-mentioned cytokines. In other instances of DDD, genetic factors or apoptosis can also cause the cells within the nucleus pulposus to emit toxic amounts of these cytokines and MMPs. In some instances, the pumping action of the disc may malfunction (due to, for example, a decrease in the proteoglycan concentration within the nucleus pulposus), thereby retarding the flow of nutrients into the disc as well as the flow of waste products out of the disc. This reduced capacity to eliminate waste may result in the accumulation of high levels of toxins that may cause nerve irritation and pain.

As DDD progresses, toxic levels of the cytokines and MMPs present in the nucleus pulposus begin to degrade the extracellular matrix, in particular, the MMPs (as mediated by the cytokines) begin cleaving the water-retaining portions of the proteoglycans, thereby reducing its water-retaining capabilities. This degradation leads to a less flexible nucleus pulposus, and so changes the loading pattern within the disc, thereby possibly causing delamination of the annulus fibrosus. These changes cause more mechanical instability, thereby causing the cells to emit even more cytokines, thereby upregulating MMPs. As this destructive cascade continues and DDD further progresses, the disc begins to bulge (“a herniated disc”), and then ultimately ruptures, causing the nucleus pulposus to contact the spinal cord or nerve root and produce pain (“sciatica”).

US Published Patent Application No. US 2003/0039651 (“Olmarker I”) teaches a therapeutic treatment of nerve disorders comprising administration of a therapeutically effective dosage of compounds, including inhibitors of MMPs.

In the examples of Olmarker I, Olmarker I further teaches that the therapeutic compounds are to be administered through systemic pathways. In particular, Olmarker I teaches that “the major contribution of TNF-α may be derived from recruited, aggregated and maybe even extravasated leukocytes, and that successful pharmacologic block may be achieved only by systemic treatment. Of note, Olmarker I appears to discourage the local addition of at least one therapeutic compound (doxycycline) to an autotransplanted nucleus pulposus to be applied to a spinal cord.

PCT Published Patent Application No. WO 02/100387 (” Olmarker II”) teaches the prevention of neovasculariation and/or neo-innervation of intervertebral discs by the administration of anti-angiogenic substances. Again, however, Olmarker II teaches systemic administration of these therapeutic agents.

U.S. Pat. No. 6,419,944 (“Tobinick I”) discloses treating herniated discs with cytokine antagonists. However, Tobinick I teaches that local administration involves a subcutaneous injection near the spinal cord. Accordingly, Tobinick does not teach a procedure involving a sustained delivery of a drug for the treatment of DDD, nor directly administering a drug into the disc.

US Published Patent Application No. 2003/0049256 (Tobinick II) discloses that injection of such therapeutic molecules to the anatomic area adjacent to the spine is accomplished by interspinous injection, and preferably is accomplished by injection through the skin in the anatomic area between two adjacent spinous processes of the vertebral column.

Tobinick II further teaches that the therapeutic compounds may be administered by interspinous injection in the human and that the dosage level is in the range of 1 mg to 300 mg per dose, with dosage intervals as short as two days. Tobinick II further discloses that other therapeutic compounds are administered in a therapeutically effective dose, which will generally be 10 mg to 200 mg per dose, and their dosage interval will be as short as once daily.

Tobinick, Swiss Med. Weekly, 2003, 133, p, 170-7 (“Tobinick III”) teaches perispinal and epidural administration of TNF inhibitors.

Karppinen, Spine, 28(8), 203, pp. 750-4, teaches intravenously injecting or orally administering infliximab into patients suffering from sciatica.

As with Tobinick I and II, Karppinen does not teach a procedure involving a sustained delivery of a drug for the treatment of DDD, nor directly administering a drug into the disc space.

U.S. Pat. No. 6,352,557 (“Ferree”) teaches adding therapeutic substances such as anti-inflammatory medications to morselized extra-cellular matrix, and injecting that combination into an interverterbral disc. However many anti-inflammatory agents are non-specific and therefore may produce unwanted side effects upon other cells, proteins and tissue. In addition, the pain-reducing effect of these agents is typically only temporary. Lastly, these agents typically only relieve pain, and are neither curative nor restorative.

Alini, Eur. Spine J. 11(Supp.2), 2002, pp. S215-220, (“Alini I”) teaches therapies for early stage DDD, including injection of inhibitors of proteolytic enzymes or biological factors that stimulate cell metabolic activity (i.e., growth factors) in order to slow down the degenerative process. Inhibitors of proteolytic enzymes constitutes a broad class of compounds, including i) inhibitors of proteolytic enzyme synthesis and ii) inhibitors of proteolytic enzyme activity. Alini I does not specify any desired types of inhibitors of proteolytic enzymes.

### SUMMARY OF THE INVENTION

The present inventors have developed a number of therapies respecting administration of IL-10 to treat spine-related inflammation and pain.

According to Brennan, Rheumatology 1999, 38, 293-7, IL-10 can induce the production of cytokine inhibitors, including the IL-1 receptor antagonist (IL-1ra) and the release of both soluble TNF receptors p55 and p75 in monocytes. Because of this utility, Brennan chartacterizes IL-10 as a ‘macrophage deactivating factor’.

According to Hart, Immunology, April 1995, 84 (4) 536-42, IL-10 and 11-4 have the capacity to downregulate both pro-inflammatory molecules TNF-α and IL-1β. Szczepanik, J. Neuroimmunology, 113(2001, 49-62 reports that IL-10 was found to suppress LPS-induced inflammatory proteins measured, including IL-1a, IL-1β, IL-6, TNF-α and MCP-1. Kelly, J. Biol. Chem. 276(49) 45564-72, reports that IL-10 antagonizes IL-1β effects.

According to Brodie, FEBS Lett., Sep. 30, 1996, 394(2), 117-20, when IL-10 contacts astrocytes, it induces the production of nerve growth factor (NGF). Therefore, IL-10 may not only inhibit inflammation associated with sciatica, it may also enhance the repair of nerves damaged by toxic cytokines associated with sciatica.

It appears that IL-10 can reduce pain. Poole, Br. J. Pharmacol., 115, 200, 684-8 reports that cytokine-mediated inflammatory hyperalgesia is limited by interleukin-10. Poole demonstrated that IL-10 both inhibited hyperalgesic cytokine release and blocked COX-2 induction. Vale, J. Pharm. Exp. Thera., 304, 2003, 102-8 reports the antinociceptive effects of IL-10 upon the writhing response in mice and zymosan-induced knee joint incapacitation in rats. Vale further hypothesizes that this pain-reducing effects may be at least partially due to the inhibition of release of proinflammatory cytokines IL-1 and TNF-a, and may also be due to a down-regulating effect in the release of eicoanoids.

It has been reported in the literature that glutamate may be present within a herniated intervertebral disc in amounts sufficient to diffuse to glutamate receptors and affect neuronal activity in the dorsal root ganglion and transmit pain. Harrington, Spine, Apr. 15, 2000, 25(8) 929-36. It has been further reported that IL-10 prevents glutamate-mediated apoptotic cell death because of the ability if L-10 to inhibit the activity proapoptotic proteins and in particular caspase-3. Bachis, J. Neuroscience, May 1, 2001, 21(9) 3104-12. Therefore, the production or administration of IL-10 may therapeutically antagonize glutamate activity associated with a herniated disc.

The concept of administering UVB light to therapeutically treat an auto-immune disease by producing autologous L-10 is disclosed in U.S. Pat. No. 5,910,309 (Ullrich). Shreedhar, J. Immunol., 1998, 160, 3783-9, suggests that UV irradiation of keratinocytes activates a cytokine cascade as follows:
PGE₂→IL-4→IL-10.

Although Ullrich appreciates the benefits of W-10, the methods of providing IL-10 disclosed by Ullrich generally concern full body irradiation producing systemic increases in L-10. Since IL-10 has a potent immunosuppressive effect, the patient receiving such treatment would be at risk of undesired side effect of having a suppressed immune system, including an increased susceptibility towards infection.

Within the nucleus pulposus of a herniated but contained disc, a degenerative cascade is occurring, wherein pro-inflammatory cytokines such as IL-1 and TNF-α are present in physiologically significant amounts. According to Ahn, Spine, May 1, 2002, 27(9), 911-7, pro-inflammatory cytokines such as IL-1 and TNF-α are present such disc tissue in physiologically significant amounts 70% and 65% of the time. However, also according to Ahn, the anti-inflammatory IL-10 is present in such disc tissue far less often (only about 9% of the time). Accordingly, it appears that contained herniations are characterized by a proinflammatory environment containing predominantly IL-1 and TNF-α.

The present inventors believe that the level of endogenous IL-10 in a contained herniation disc tissue is generally insufficient to antagonize pro-inflammatory molecules therein.

Therefore, a first aspect of this invention relates to a method of treating low back pain, wherein an effective amount of anti-inflammatory cytokine IL-10 is injected into a degenerating disc.

Therefore, in accordance with the present invention, there is provided a method of treating degenerative disc disease in an intervertebral disc of a patient having a nucleus pulposus and an annulus fibrosus, comprising the steps of:
a) intradiscally administering an effective amount of a formulation comprising interleukin-10 (IL-10) into the intervertebral disc.

Therefore, in accordance with the present invention, there is provided a method of treating sciatica, comprising the steps of:
a) epidurally administering an effective amount of a formulation comprising interleukin-10 (IL-10) into the vicinity of a nerve root.

IL-10 may be produced by exposing macrophages to an effective amount of UV radiation. Ullrich, Photochem. Photobiol. 1996, 64(2), 254-8 reports that Th2 cells may be activated by UV radiation to emit IL-10.

Therefore, a second aspect of this invention also relates to a method of treating low back pain, wherein macrophages and/or lymphocytes are obtained from the patient (such as through their isolation from centrifuged blood or bone marrow), the isolated cells are exposed to UVB radiation, and the UVB-activated cells are injected into a degenerating disc or in the vicinity of a nerve root.

Therefore, in accordance with the present invention, there is provided method of administering interleukin-10 (IL-10) to a patient, comprising:
a) obtaining from the patient cells viable capable of producing IL-10;
b) exposing the viable cells to UV-B light for a period sufficient to produce induced cells, and
c) administering the induced cells to a location in the patient, whereby the induced cells in vivo produce IL-10 at the location.

This aspect of the invention has the advantage of essentially irradiating only macrophages and/or lymphocytes, whose half-life is believed to be on the order of a month.

Park, Spine, 27(19), 2125-28, 2002, reports that intervertebral disc cells were stained positively for CD4 Tcell antibody, thereby identifying disc cells as a source of Th1 and Th2 cytokines. Whereas activated Th1 cells contribute to a cellular immune response and emit pro-inflammatory cytokines such as IFN-gamma and IL-2, Th2 cells contribute to a humoral immune response and emit anti-inflammatory cytokines, including IL-10. Although Park concludes that disc cells are preferentially expressing Th2 cytokines, the present inventors note the low levels of IL-10 reported by Ahn and believe that UV irradiation of the cells within the disc will lead to an enhanced production of IL-10 in an amount sufficient to antagonize both Il-1β and TNF-α.

Therefore, a third aspect of this invention also relates to a method of treating low back pain, wherein CD4-type disc cells (as well as endogenous macrophages and lymphocytes that have accumulated within the contained nucleus pulposus) are irradiated by an intradiscal UV-B light probe with an amount of UV-B light effective to activate the Th2 pathway. These activated migratory cells then locally produce IL-10 within at least the nucleus pulposus portion of the disc, and the IL-10 then attenuates the pro-inflammatory reaction within the disc.

A fourth aspect of this invention also relates to an implant for treating DDD, wherein the implant is adapted to fit within the disc space and comprises a UV-B LED and an antenna. In use, endogenous macrophages and lymphocytes or disc cells that have accumulated within the inflamed disc are irradiated by the UV-B light implant with an amount of UV-B light effective to activate the Th2 cell pathway. These activated migratory cells then locally produce IL-10 within at least the nucleus pulposus portion of the disc, and the IL-10 then attenuates the pro-inflammatory reaction within the disc.

Accordingly, in some embodiments of the present invention, there is provided a method of treating low back pain, comprising the steps of:
a) providing an implant a UVB light source,
b) positioning the implant within the nucleus pulposus of an intervertebral disc, and

c) activating the light source to irradiate the disc with an amount of UVB light sufficient to activate cells therein to produce IL-10.

Therefore, in accordance with the present invention, there is provided an intervertebral implant comprising:
a) an ultraviolet light LED, and
b) an antenna in electrical connection with the LED.

In some patients, disc degeneration proceeds to the point wherein a tear is produced in the annulus fibrosus. Following this injury, the blood vessels present on the outer surface of the annulus fibrosus invade the tear as part of the healing process. This microvasculature also brings with it macrophages and lymphocytes. These macrophages and lymphocytes emit pro-inflammatory molecules such as IL-1β, and TNF-α. Due to the presence of nociceptors in the periphery of the annulus fibrosus, these pro-inflammatory molecules may also cause pain.

Therefore, a fifth aspect of this invention also relates to a method of treating low back pain, wherein endogenous macrophages and lymphocytes that have accumulated around blood vessels present in the outer portion of the annulus fibrosus are irradiated by a UV-B light probe with an amount of UV-B light effective to activate them to convert to the Th2 pathway. These activated migratory cells then locally produce IL-10 within at least the annulus fibrosus portion of the disc, and the IL-10 then attenuate the inflammatory reaction within the disc.

Therefore, in accordance with the present invention, there is provided a method of treating DDD in an intervertebral disc, comprising the steps of:
a) providing a probe having a distal end portion having a UV light source,
b) positioning the UV light source within the disc, and
c) activating the light source to irradiate the disc in an amount of UV light sufficient to activate macrophages therein.

Also in accordance with the present invention, there is provided a method of treating DDD in an intervertebral disc having a tear in an annulus fibrosus, comprising the steps of:
a) providing a probe having a distal end portion having a UV light source,
b) positioning the UV light source adjacent the tear, and
c) activating the light source to irradiate the disc in an amount of UV light sufficient to activate macrophages present in the vicinity.

As mentioned above, DDD often progresses to the point where the annulus fibrosus tears and the nucleus pulposus exudes from the tear and often reaches a nerve root adjacent the disc. Once extruded, the surface of the nucleus pulposus becomes populated with small blood vessels that are paths for the invasion of macrophages and lympocytes. These macrophages and lymphocytes recognize the nucleus pulposus as foreign and begin emitting pro-inflammatory cytokines. It is believed that these pro-inflammatory cytokines irritate the nerve root and cause sciatic pain. According to Park, supra, the T cell cytokine distribution in the extruded nucleus pulposus turns from Th2 type anti-inflammatory cytokines to Th1 type pro-inflammatory cytokines. For example, whereas the intra-nucleus puposus concentrations of pro-inflammatory molecules IL-12 and INF-gamma double or triple upon extrusion, the concentration of anti-inflammatory IL-4 decreases by over about 90%.

The present inventors have noted that the autologous cells that produce IL-10 when activated by UV-B radiation (macrophages and lymphocytes) are the same cells that participate in the inflammatory response characteristic of sciatica. Therefore, it appears that a great concentration of cells capable producing of IL-10 reside precisely within region of undesired inflammation. Therefore, it is believed that sciatica may be effectively treated by irradiating the inflamed region with UV-B light, thereby suppressing the pro-inflammatory (Th1) cells in that region that are emitting pro-inflammatory cytokines and activating the IL-10 emitting (Th2) cells and macrophages.

It has been reported by Autio, Spine, 2004, 29(15), 1601-7, that administration of steroids not only provides anti-inflammatory relief, it also enhances resorption of a herniated nucleus pulposus. Therefore, attenuatation of the inflammatory response may also enhance resorption.

Therefore, a sixth aspect of this invention also relates to a method of treating sciatica, wherein endogenous macrophages and lymphocytes that have accumulated around the exuded nucleus pulposus are irradiated by a UV-B light probe with an amount of UV-B light effective to activate the Th2 pathway and locally produce IL-10 in the vicinity of the exuded nucleus pulposus. The IL-10 then attenuates the inflammatory reaction around the nerve root and stops the pain by antagonizing IL-1β and TNF-α.

Therefore, in accordance with the present invention, there is provided a method of treating sciatica, comprising:
a) providing a probe having a distal end portion having a UV light source,
b) positioning the UV light source in the vicinity of the exuded nucleus pulposus, and
c) activating the light source to irradiate the exuded nucleus pulposus in an amount of UV light sufficient to activate macrophages present in the vicinity.

A seventh aspect of this invention relates to a method of treating low back pain, wherein facet joint synovium (as well as endogenous macrophages and lymphocytes that have accumulated within the synovium) is irradiated by an interfacet UV-B light probe with an amount of UV-B light effective to activate the Th2 pathway. These activated migratory cells then locally produce IL-10 within at least the synovium of the facet joint, and the IL-10 then attenuates the inflammatory reaction within the facet joint.

Therefore, in accordance with the present invention, there is provided a method of treating a facet joint, comprising:
a) providing a probe having a distal end portion having a UV light source,
b) positioning the UV light source within a synovium of the facet joint, and activating the light source to irradiate the synovium in an amount of UV light sufficient to activate macrophages and lympocytes present in the synovium.

### DESCRIPTION OF THE FIGURES

FIG. 1 discloses the injection of IL-10 from a syringe into the nucleus pulposus of a degenerating disc.

FIG. 2a discloses a device comprising a syringe and a UV-B light source, wherein the UV-B light source irradiates a buffy coat containing macrophages to activate the macrophage to the Th2 pathway and produce IL-10.

FIG. 2b discloses a syringe having an etched glass inner barrel. The etched glass will attract IgG complexes, which will bind macrophages. The presence of IgG will enhance the rate of IL-10 production from the activated macrophages.

FIGS. 2c-2e show how macrophages are accumulated on the surface of the etched glass via IgG complexes.

FIG. 2f shows a layer of immobilized IgG within the syringe.

FIG. 3 discloses an intradiscal probe having a rotating distal UV-emitting head within the nucleus pulposus of a degenerating disc.

FIG. 4 discloses an externally powered UV-B light implant that produces IL-10 in vivo and on demand.

FIG. 5 discloses a method of treating low back pain, wherein a UV probe is placed outside the disc in a position adjacent a tear in the annulus fibrosus.

FIG. 6 shows a method of treating sciatica, wherein endogenous macrophages that have accumulated around the exuded nucleus pulposus are irradiated by a UV-B light probe with an amount of UV-B light effective to activate the endogenous macrophages to the Th2 pathway and to locally produce IL-10 in the vicinity of the exuded nucleus pulposus.

FIG. 7 is a schematic graph of IRAP and IL-10 concentrations produced by dual activation of a coated UV probe.

FIG. 8a is a plan view of a coated UV probe of the present invention.

FIGS. 8b-d show the probe of FIG. 8a inserted into an extruded nucleus pulposus, into a facet joint and into a contained nucleus pulposus.

FIG. 9 shows a UV probe of the present invention having a light port.

FIG. 10a shows a UV probe having three distal tynes.

FIG. 10b shows a UV probe having a webbed distal portion.

FIGS. 10c and d show a UV probe having a dried and swollen collagen collar thereon.

FIG. 11 shows a probe having a coating thereon capable of inducing macrophages to emit anti-inflammatory cytokines.

### DETAILED DESCRIPTION OF THE INVENTION

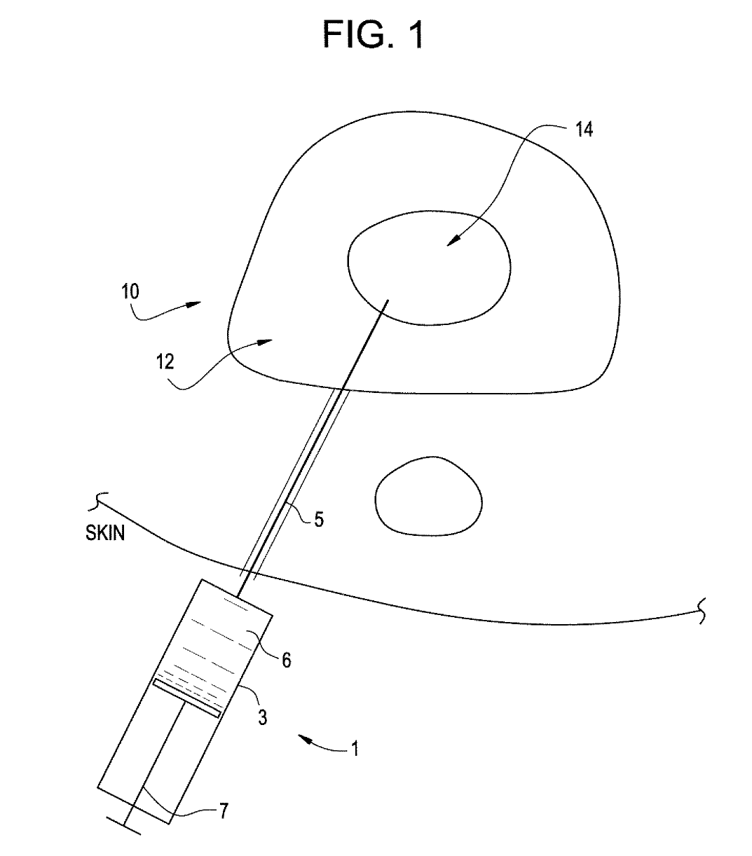

In the first aspect of this invention an effective amount of anti-inflammatory cytokine IL-10 is injected into a degenerating disc. Now referring to FIG. 1, there is provided a syringe 1 having a barrel 3 containing a formulation 6 comprising an effective amount of IL-10. A needle 5 extends from the syringe through annulus fibrosus 12 into a nucleus pulposus region 14 of a patient's disc 10. When plunger 7 is depressed, the IL-10 formulation is injected into the nucleus pulposus 14.

Preferably, the injection provides an in vivo IL-10 concentration of at least 0.1 ng/ml, more preferably at least 1 ng/ml, more preferably at least 10 ng/ml.

Preferably, the injection volume is between 0.1 and 1 cc. Preferably, the IL-10 concentration in the injection is at least 0.1 ng/ml, more preferably at least 1 ng/ml, more preferably at least 10 ng/ml.

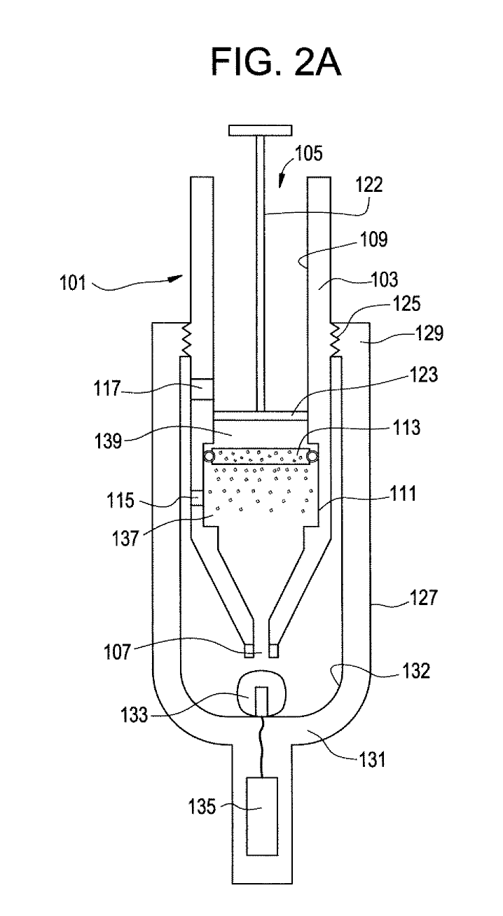

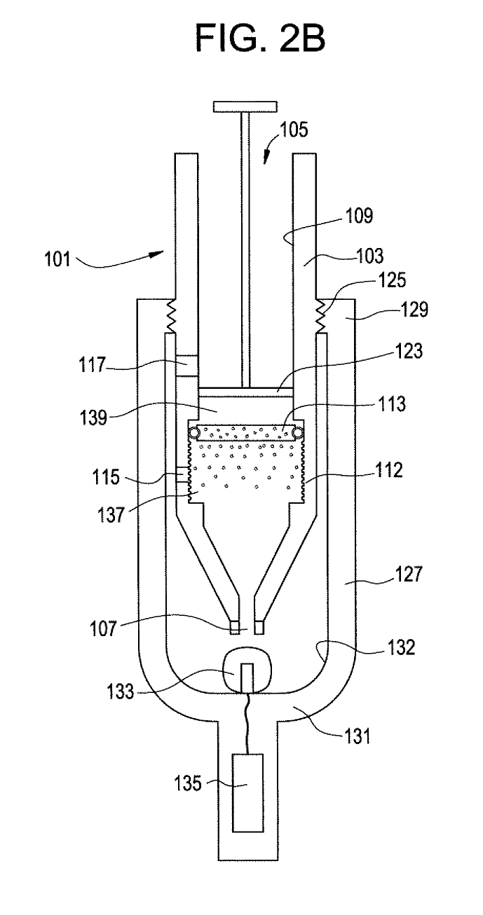

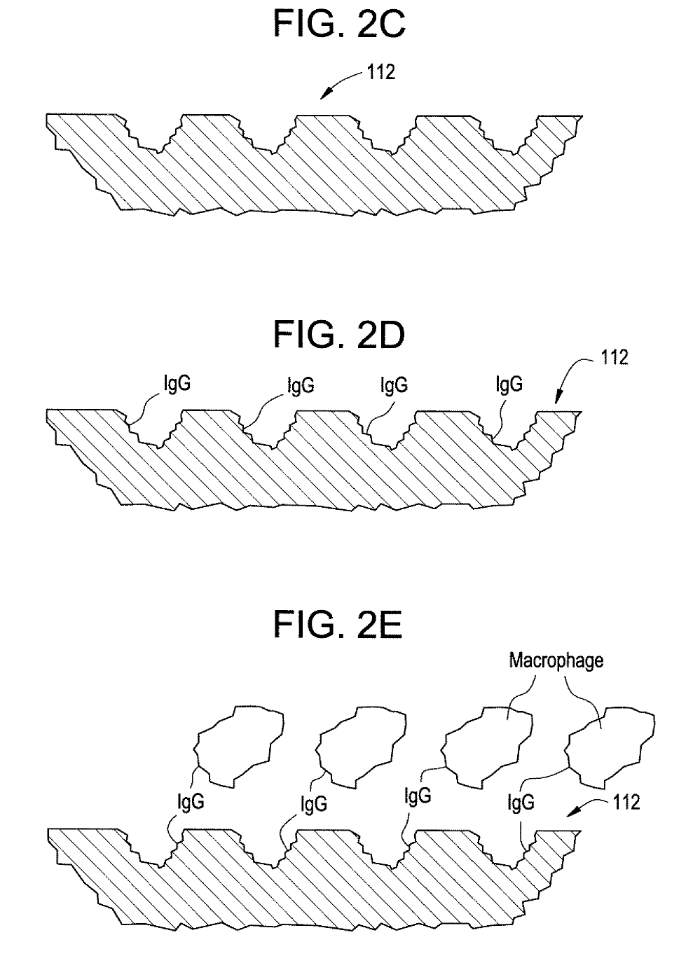

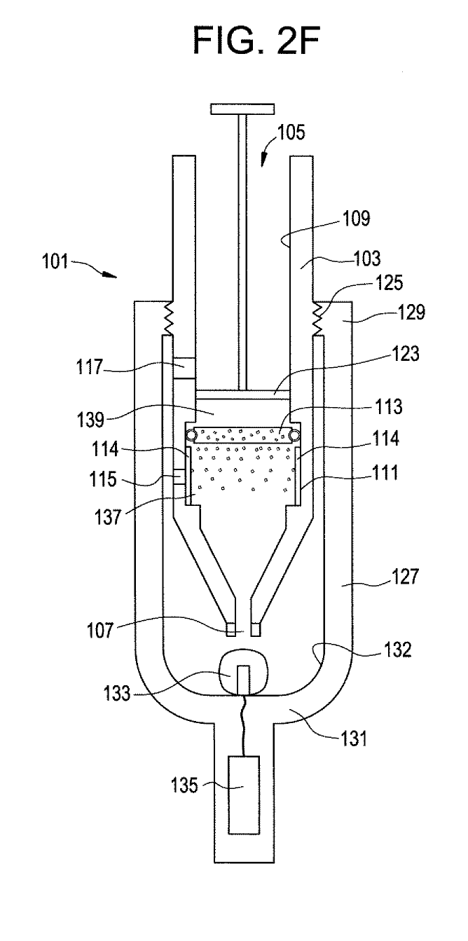

Now referring to FIG. 2a, there is provided a syringe 101 adapted for inducing and delivering viable cells of the present invention. This syringe is adapted to receive concentrated Th2 cells or macrophages, dewater the cells, receive UVB light, and finally deliver the induced cells to the patient.

The syringe comprises a barrel 103 having an inner wall 109, a proximal open end 105 and a distal open end 107. A recess 111 is provided in a portion of the inner wall forming chamber 137 in order to accommodate axial sliding of moveable filter 113. The syringe further has side ports 115 and 117 having gaskets (not shown). The syringe further include a plunger 122 having a distal plug 123, and a threaded portion 125 adapted for threadable connection to a UVB source.

The apparatus as shown further includes a UV source 127 adapted for connection to the syringe 101. The purpose of the UV source 127 is to reliably produce an appropriate dose of UV radiation to the induced cells. The UV source has a threaded end 129 adapted for threadable connection with the corresponding thread 125 on the outer surface of the syringe 101. The UV source has a closed end 131 having an inner surface 132 having a cup shape which houses a UV light 133 connected to an energy source 135. The inner surface is preferably made of a reflective material to direct the UV light towards the induced cells, while cup shape of the inner surface also direct the UV light towards the induced cells.

In use, the clinician adds the concentrated Th2 cells or macrophages to the chamber 137 defined by the syringe barrel 103 and filter 113. Next, the UV source is threaded onto the syringe and the UV source is activated to irradiated the cells with an effective amount of UV light. The clincian then waits up to two hours so that the inducer may induce the cells. After two hours, the inducing fluid is washed away, optionally by adding saline to the chamber through port 115. Next, plunger 122 is partially withdrawn from the barrel 103, thereby creating a vacuum and drawings fluid from the chamber 137 into space 139. A needle is then inserted into space 139 through port 117 in order to remove the withdrawn fluid.

Next, cryoprecipitate fibrinogen and thrombin are added to the chamber through port 115 in order to begin the clotting process.

Lastly, the plunger 122 is advanced so that the contents of the chamber 137 are injected into the intervertebral disc.

Because, in some embodiments, the induced cells are immediately injected into the patient so that the patient serves as the incubation receptacle for the induced cells, there is no need to wait for ex vivo production of IL-10. Accordingly, in preferred embodiment, the induced cells are injected into the disc less than 10 hours after the mixing step, more preferably less than 5 hours, more preferably less than 3 hours.

In some embodiments, the smooth recessed portion of the inner wall 109 of the syringe 101 of FIG. 2a may be etched 112, as in FIG. 2b. Now referring to FIG. 2c, there is provided a cross section of a magnified view of the etched glass wall surface of FIG. 2b. In FIG. 2d, native IgG from the patient's blood attaches to the surface 112. In FIG. 2e, native macrophages attach to the native IgG. The IgG-macrophage complex will produce IRAP. Accordingly, in these embodiments, the syringe of FIG. 2b produces both IL-10 (derived from the UV irradiation of the Th2 cells or macrophages and IRAP (derived from the adhesion of IgG-macrophage complexes to the inner wall).

In some embodiments, and now referring to FIG. 2f the etched glass element 112 of FIG. 2b is replaced with an immobilized inducer coating 114, such as immobilized IgG or IgA.

Shreedhar, J. Immunol., 1998, 260, 3783-9, reports producing 313 pg IL-10/ml by exposing keratinocytes to 0.02 J/cm² UV light, while Kang, J. Immunol., 1994, 153, 5256 reports producing 333 pg/ml in a supernatant from CD11b+ macrophages exposed to 4 MEDs of UV light. Therefore, in one embodiment, it may be anticipated that the clinician may be able to produce a 1 cc bolus capable of generating about 0.3 ng of IL-10 with as little as 0.02 J/cm² UVB light. It is believed that higher does of UVB light will produce correspondingly greater amounts of IL-10.

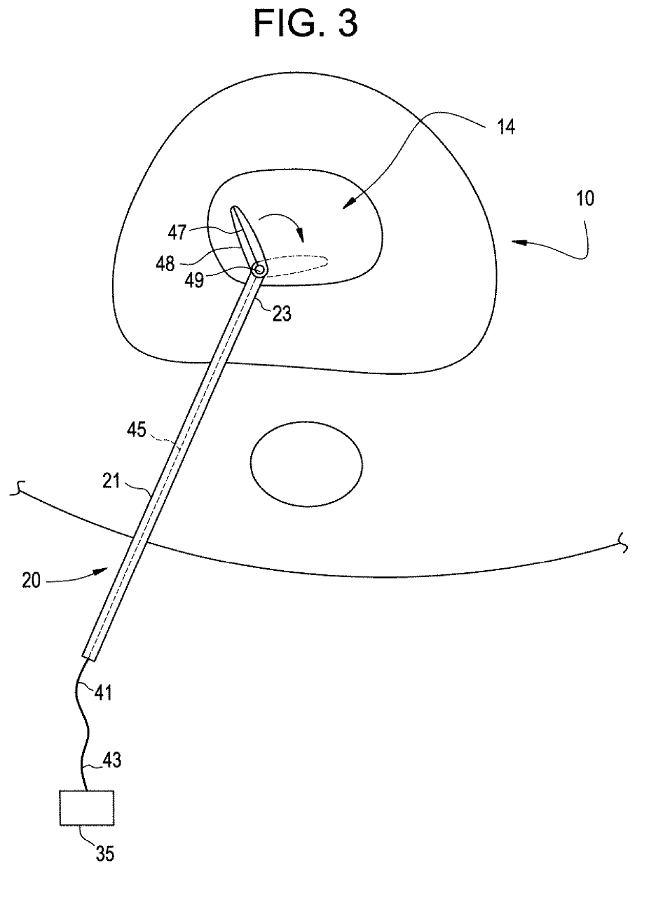

Now referring to FIG. 3, there is provided a UV probe 20 of the present invention, comprising:
a) a tubular jacket 21 having an inner bore and a distal end 23,
b) a UV transparent flange 48 rotatably connected to the distal end of the tubular jacket 21,
c) a UVB light source 35, and
d) a fiber optic cable 41 having a proximal portion 43 attached to the UVB light source 35, a distal portion 45 passing through the tubular jacket 21 and a distal end 47 attached to the flange 48.

In this embodiment, the clinician inserts the distal end of the probe into the nucleus pulposus portion of the disc and activates the probe. The clinician then adjusts the position of the flange through pin 49 so that the distal end of the fiber optic moves to a new position. The clinician then re-activates the UVB light source to impart a second effective dose of UV radiation upon a new region of tissue.

Accordingly, the ability to move the position of the distal end of the UV fiber optic cable in this FIG. 3 embodiment has the advantage of increasing the volume of nucleus pulposus exposed to an effective amount of UVB radiation.

In some embodiments, the light source is provided on the implant and is adapted to be permanently implanted into the patient. The advantage of the internal light source is that there is no need for further transcutaneous invasion of the patient. Rather, the internally-disposed light source is activated by either a battery disposed on the implant, or by telemetry, or both. In some embodiments of the present invention using an internal light source, the light source is provided by a bioMEMs component. In one embodiment thereof, the internal light source comprises a UV light source, and preferably comprises an AlGaN substrate. It has been reported by Stutzmann, Diamond and Related Materials, 11 (2002) 886-891, that AlGaN may have future application as a biosensor. Stutzman further conducted studies on the biocompatibility of GaN, AlGaN and AlN, and found very little interaction with living cell tissue, thereby suggesting the biocompatibility of these materials.

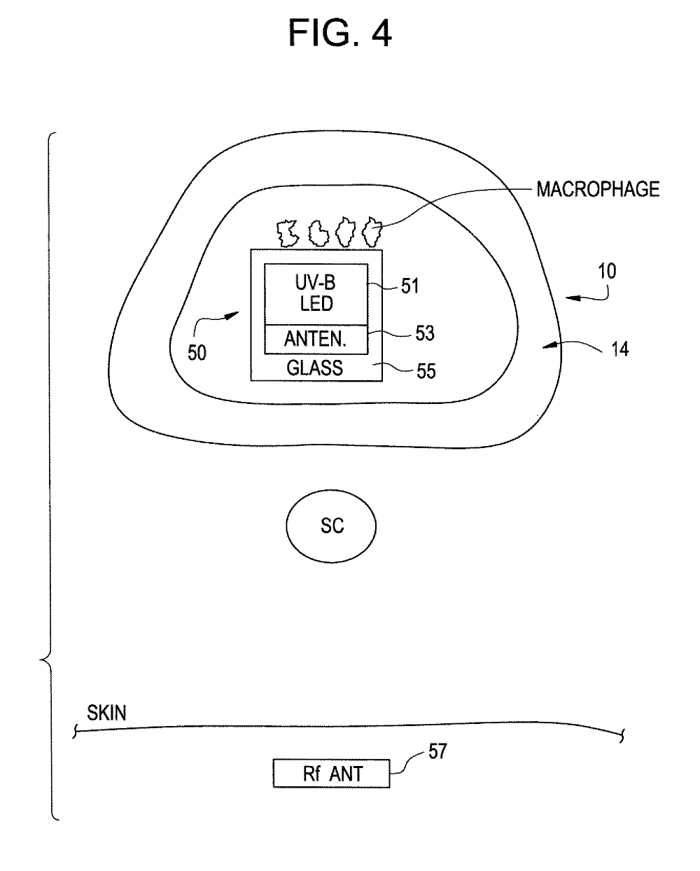

Now referring to FIG. 4, there is provided an implant 50 for treating a degenerating disc, comprising:
a) a UV-B Light emitting diode (LED) 51, and
b) an antenna 53 in electrical connection with the LED.

In order to protect the active elements of the device from the nucleus pulposus 14, in some embodiments, and again referring to FIG. 4, the UV LED is encased in a casing 55, in this case, glass, for example. This casing both protects the LED components from the nucleus pulposus, and also prevents the LED components from elicting an immune reaction In some embodiments, the casing is made of a UVB transparent material. The UV transparent material may be placed adjacent the LED component so that UV light may be easily transmitted therethrough. In some embodiments, the UV transparent casing is selected from the group consisting of silica, alumina and sapphire. In some embodiments, the light transmissible material is selected from the group consisting of a ceramic and a polymer. Suitable UV-transmissible ceramics include alumina, silica, CaF, titania and single crystal-sapphire. Suitable light transmissible polymers are preferably selected from the group consisting of polypropylene and polyesters.

In use, the clinician inserts the implant 50 into the nucleus pulposus portion 14 of an inflamed disc. After closing the patient, the clinician then imparts Rf energy to the implant 50 via a suitable antenna 57.

In one embodiment, there is provided an exemplary UV unit having an internal light source, an externally based-control device having an RF energy source, and an antenna for transmitting signals to the internally-based antenna provided on the implant. These antennae may be electro-magnetically coupled to each other. The internal antenna sends electrical power to the light emitting diode (LED) disposed internally on the implant in response to the transmitted signal transmitted by the external antenna. The light generated by the LED travels across the UV transparent casing and into the disc tissue.

In other embodiments, the implant has an internal light source further contains an internal power source, such as a battery (which could be re-chargeable), which is controlled by an internal receiver and has sufficient energy stored therein to deliver electrical power to the light source in an amount sufficient to cause the desired light output.

When the implant is coupled with external energy, power can be transmitted into the internal device to re-charge the battery.

In some embodiments, the light generated by the implant is powered by wireless telemetry integrated onto or into the implant itself. In one embodiment, the LED may comprise a radiofrequency-to-DC converter and modulator. When radiofrequency signals are emitted by the external antenna and picked up by the internal antenna, these signals are then converted by the receiver into electrical current to activate the light source of the PCO unit.

In one embodiment, the implant may have an internal processor adapted to intermittently activate the LED.

In some embodiments, the telemetry portion of the device is provided by conventional, commercially-available components. For example, the externally-based power control device can be any conventional transmitter, preferably capable of transmitting at least about 40 milliwatts of energy to the internally-based antenna. Examples of such commercially available transmitters include those available from Microstrain, Inc. Burlington, V. Likewise, the internally-based power antenna can be any conventional antenna capable of producing at least about 40 milliwatts of energy in response to coupling with the externally-generated Rf signal. Examples of such commercially available antennae include those used in the Microstrain Strainlink™ device. Conventional transmitter-receiver telemetry is capable of transmitting up to about 500 milliwatts of energy to the internally-based antenna.

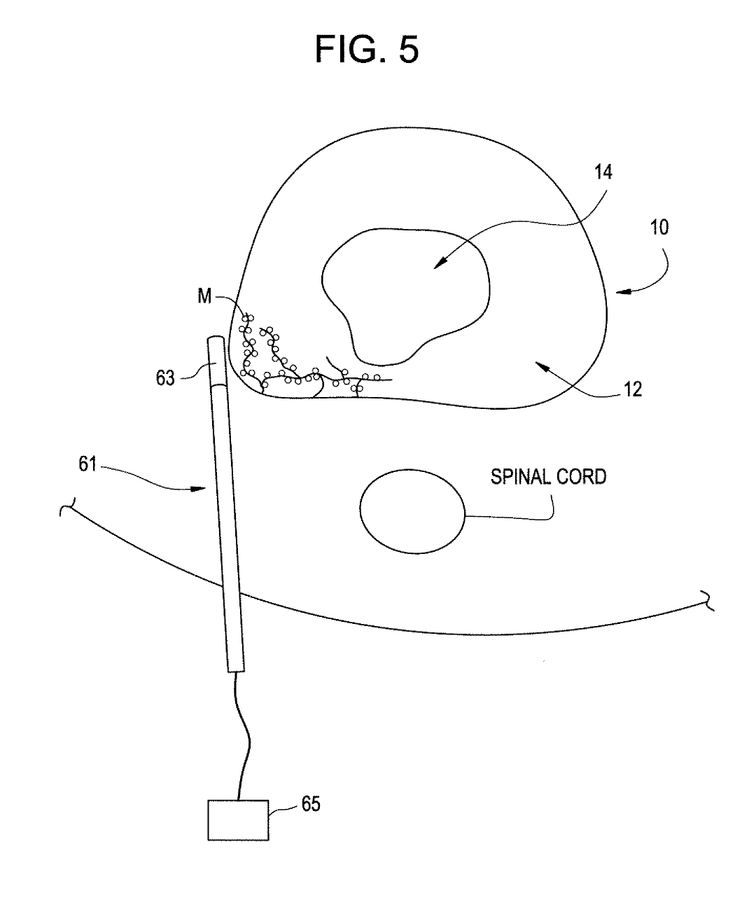

Now referring to FIG. 5, there is provided a UV probe 61 of the present invention substantially similar to that of FIG. 3, except that the rotatable flange is eliminated, thereby fixing the distal end 63 of the fiber optic.

As seen in FIG. 5, when a portion of the annulus fibrosus 12 becomes tom, migrating macrophages M and lymphocytes invade the area of the tear. These cells begin to emit pro-inflammatory cytokines such as TNF-α. These pro-inflammatory cytokines then contribute to both inflammation and pain (due to contact with nociceptors present within the peripheral region of the annulus fibrosus).

When probe 61 is inserted into the area of the annular tear and light source 65 is activated, UVB light is emitted from the distal end 63 of the fiber optic cable. This light causes activation of local macrophages and Th2 lympocytes to produce an effective amount of IL-10. This IL-10 then antagonizes the pro-inflammatory cytokines to reduce or eliminate inflammation and pain.

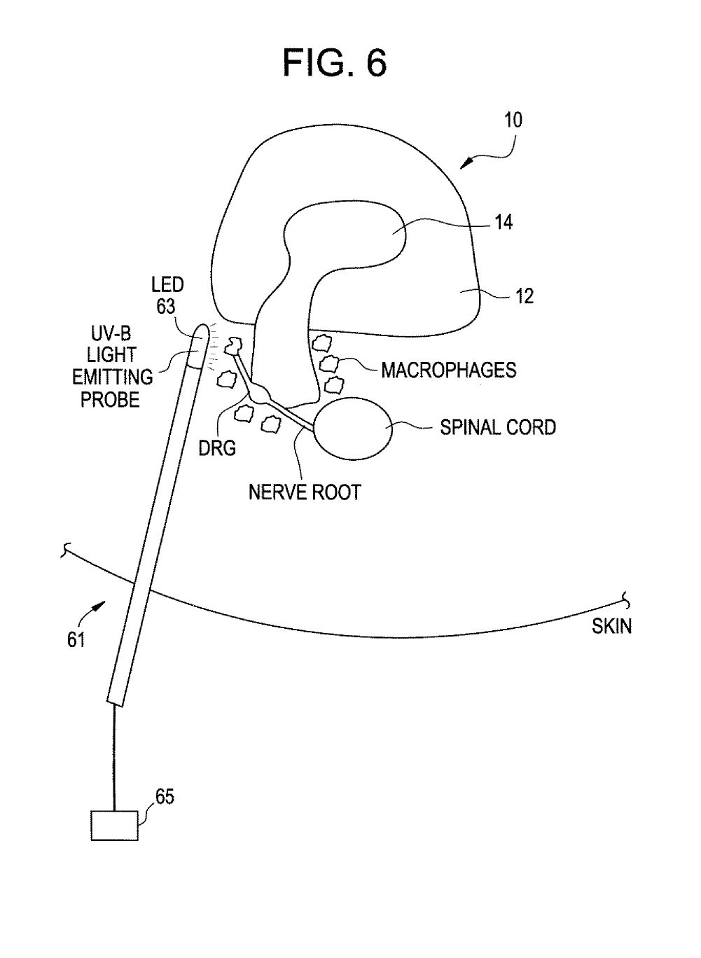

Now referring to FIG. 6, there is provided a UV probe 61 of the present invention substantially similar to that of FIG. 5.

As seen in FIG. 6, when a portion of the annulus fibrosus 12 becomes ruptured, migrating macrophages M and lymphocytes invade the area of the rupture and attach themselves to the rim of the exuded nucleus pulposus 14. These cells begin to emit pro-inflammatory cytokines such as TNF-α. These pro-inflammatory cytokines then contribute to both inflammation and pain (due to contact with the sciatic nerve root present in the vicinity of the rupture).

When probe 61 is inserted into the area of the annular tear and light source 65 is activated, UVB light is emitted from the distal end 63 of the fiber optic cable. This light causes activation of macrophages and Th2 lympocytes present on the rim of the nucleus pulposus 14 to produce an effective amount of IL-10. This IL-10 then antagonizes the pro-inflammatory cytokines to reduce or eliminate inflammation and pain.

In some embodiments, the sciatica therapy may be both diagnostic and therapeutic. In some embodiments, a cannulated catheter having a bore and an outer collar containing a fiber optic could be designed so that the clinician could inject either saline or an analgesic through an inner bore and deliver UV-B light through its outer collar. The clinician could place the catheter in the epidural space over the dorsal root ganglia (DRG), and provide a radio-opaque dye through the bore to confirm the position of the catheter tip. See FIG. 6. Next, the clinician could inject an analgesic through the bore (such as bupivicaine) as a diagnostic block. If the analgesic effect confirms the appropriate position of the fiber optic, UVB treatment would then be provided.

In some embodiments, the cannulated catheter could to have an outer diameter of about 3 mm.

When a facet joint begins to degenerate, migrating macrophages M and lymphocytes invade the synovium portion of the joint even without a rupture of the joint These cells begin to emit pro-inflammatory cytokines such as TNF-α a. These pro-inflammatory cytokines then contribute to both inflammation and pain (due to contact with the nociceptors present within the joint).

When a probe of FIGS. 3, 5 or 6 is inserted into the synovium and the light source is activated, UV light is emitted from the distal end of the fiber optic cable. This light causes activation of macrophages and Th2 lympocytes present within the synovium to produce an effective amount of IL-10. This IL-10 then antagonizes the pro-inflammatory cytokines to reduce or eliminate inflammation and pain.

In some embodiments, the light source has a spectral maximum in the range of the UVB components of the solar spectrum. Preferably, the light source has a spectral maximum in the range of less than about 380 nm, and is preferably between 280 nm and 320 nm. In some embodiments, the light source has a spectral maximum of between about 300 nm and 315 nm.

In some embodiments, the light source is situated to irradiate adjacent cells with between about 0.02 J/cm² and about 10 J/cm² energy, preferably between about 1 J/cm² and 10 J/cm². Without wishing to be tied to a theory, it is believed that light transmission in this energy range will be sufficient to activate the disc cells, macrophages or Th2 lymphocytes present within adjacent tissue. Shreedhar, J. Immunol., 1998, 160, 3783-9 has reported using a light dose of 0.02 J/cm² in order to activate keratinocytes to produce IL-10. Schmitt, J. Immunlology, 2000, 165:3162-7 has reported using a dose of 1.5 J/cm². Rivas, J. Immun, 149, 12, 1992, 3865-71 has reported using a dose of 0.02 J/cm². Therefore, it is believed that irradiating inflamed brain tissue with at least about 0.02 J/cm² of UV radiation will induce the macrophages and Th2 cells therein to produce and emit IL-10.

In some embodiments, the light source is situated to produce an energy intensity at the cell surface of between 0.1 watts/cm² and 10 watts/cm². In some embodiments, the light source is situated to produce about 1 milliwatt/cm². This latter value has been reported by Ullrich to effectively irradiate a cell surface in an amount sufficient to produce IL-10.

It is well known in the art that DDD is characterized not only by the presence of macrophages, but also the presence of lymphocytes. For example, Virri, Spine, November 1, 26(21) 2311-5 reports that 17% of examined discs contained abundant T cells, 17% of examined discs contained activated T cells, 16% of examined discs contained abundant B cells, and 34% of examined discs contained abundant macrophages. Since it is also known that lymphocytes may secrete pro-inflammatory cytokines via the Th1 pathway, it is appropriate to consider the activity of these T cells in DDD therapies.

U.S. Pat. No. 6,083,919 (Johnson) has reported that co-administration of IL-10 and TGF-β in amounts effective to produce a synergistic reduction in lymphocyte activity. In one example, Johnson reports that about 0.3 ng/ml of each of IL-10 and TGF-β inhibits the activation of self-reactive T cells in autoimmune diseases from 20,000 units to less than 500 units.

Therefore, in accordance with the present invention, there is provided a method of treating DDD, wherein both IL-10 and TGF-B are intradiscally admininstered in amounts effective to produce a synergistic reduction in lymphocyte activity.

In one embodiment of the present invention, effective amounts of TGF-β can be obtained by activation of platelet-rich plasma (PRP). Preferably, the TGF-β is administered to provided an effective concentration of at least 1 ng/ml.

The present inventors have also noted that, in addition to UVB light, certain biomaterial surfaces also possess abilities to induce macrophages to produce anti-inflammatory compounds.

For example, Meijer, Inflammatory Research 52 (2003) 404-407, teaches a method of producing TRAP in whole blood samples in therapeutically relevant amounts by its physico-chemical induction in a syringe. Meijer further reports producing up to 8 ng IRAP/ml blood by such methods and states that a minimum IL-1/IRAP ratio of 1:10 is required to inhibit IL-1 activity. U.S. Pat. No. 6,623,472 (“Reinecke I”) teaches the production of autologous IRAP by contacting blood with a syringe having an inner structure coated with immunoglobulin G. Reinecke I teaches that the IRAP produced by this method may be injected into an intervertebral disc to treat neurologically-caused back complaints. U.S. Pat. No. 6,713,246 (“Reinecke II”) teaches the production of autologous IRAP by contacting blood with a syringe having an etched inner barrel, and also that the IRAP may be injected into an intervertebral disc. Reinecke further teaches that this method requires waiting at least 12 hours (and preferably 24 hours) for the viable cells to incubate in the syringe to produce IRAP.

Each of Meijer, and Reinecke I and II teach ex vivo production of RAP. However, it has been observed by the present inventors that Meijer reports that the physical induction processes produce physiologically significant amounts of IRAP within a fairly short time period. For example, Meijer reports that a mere half-hour incubation of its syringe increases the IRAP concentration therein from 78 pg/ml to between 778-2000 pg/ml. Meijer further reports that six-hour incubation of its syringe increases the IRAP concentration therein from 78 pg/ml to between 5000 pg/ml. Similarly, Takeda, Inflamation Research, 52(2003, 287-290, reports the production of about 1.5 ng/ml of IRAP from blood incubated for 2 hours with cellulose acetate beads.

Therefore, because the induction of IRAP appears to occur so quickly, the present inventors envision an in vivo physical induction of IRAP by selected probes in environments containing elevated amounts of macrophages or Th2 lymphocytes.

Therefore, a seventh aspect of this invention relates to a method of treating low back pain, wherein a probe have an IRAP-inducing surface is inserted into a facet joint synovium for a time sufficient to activate the Th2 pathway from endogenous macrophages and lymphocytes that have accumulated within the synovium. These activated migratory cells then locally produce IRAP within at least the synovium of the facet joint, and the IRAP then attenuate the inflammatory reaction within the facet joint.

Therefore, a eighth aspect of this invention relates to a method of treating sciatica, wherein a probe have an IRAP-inducing surface is inserted into an extruded nucleus pulposus located in the vicinity of a nerve root for a time sufficient to activate the Th2 pathway from endogenous macrophages and lymphocytes that have accumulated on the surface of the extruded nucleus pulposus. These activated migratory cells then locally produce IRAP within at least the nucleus pulposus, and the IRAP then attenuates the inflammatory reaction around the nerve root.

It is believed that each of the following coatings can induce the production of either IRAP or IL-10 from the macrophage-filled environments of sciatica and an inflamed facet joint.

U.S. Pat. No. 6,623,472 (“Reinecke I”) teaches the production of autologous IRAP by contacting blood with a syringe having an inner structure coated with immunoglobulin G. According to Takeda, when human monocytes were cultured on an IgG coated surface in the absence of LPS, the cells produced a small amount of IL-1B but a large amount of IRAP. Takeda then concludes that IgG adsorption onto the beads is necessary for IRAP release, probably by mediating neutrophil and monocyte adsorption to the beads which then release IRAP.

Mosser, J. Leukocyte Biology, 72, 2003, 209 reports that IL-4 is thought to induce macrophages to produce large amounts of Il-10 in a process known as “alternative activation”.

As noted above, U.S. Pat. No. 6,713,246 (“Reinecke II”) teaches the production of autologous IRAP by contacting blood with a syringe having an etched inner barrel.

According to Takeda, plasma protein like IgG adsorb onto CA and mediate complement activation locally, generating active fragments C3A, C5a, C3bi and others. C3a and C5a can activate leukocytes, while C3bi adsorbs onto CA beads and together with IgG mediate granulocyte and monocyte adhesion to the beads via the complement receptor 3 and FcY receptor.

The literature has reported that IgA is a very promising inducer of IRAP. The literature has reported that IgA not only induces the production of IRAP in monocytes (Wolf I, Clin. Exp. Immunology, 1996, 105:537-543), it also reported that IRAP also downregulates TNF-α (Wolf II, Blood, 83(5) Mar. 1, 1994, pp. 1278-88). Moreover, Wolf I reports that the induction of human monocytes by IgA raised the IRAP level in the culture from <1 ng/ml to over 65 ng IRAP/ml in 24 hours.

It is known in the literature that poly I:C induces macrophages to produce interferon alpha.

In some embodiments, a probe of the present invention contains both a UV device and a coating adapted to induce macrophages to emit an anti-inflammatory cytokine (such as IRAP, IL-10 or interferon).

In preferred methods of use, the dual probe is first inserted into the region of interest; the UVB light is then activated for a first time; a refractory period then ensues wherein macrophage or lymphocyte induction via the surface coating is allowed to proceed, and finally the UVB light is activated for a second time. This invention arises from the observation that UVB irradiation not only generally takes on the order of minutes, but also that macrophage/lymphocyte production of a cytokine is typically more efficient if the induction environment is already primed with that cytokine. Therefore, the skilled artisan gets the benefit of not just one phase of IRAP production but of three:
a) a first production phase wherein UVB light activates Th2 macrophages to emit IL-10;
b) a second production phase wherein the treated surface induces Th2 macrophages to emit either IRAP or IL-10; and
c) a third production phase wherein UV light activates primed Th2 macrophages to emit large amounts of IL-10.

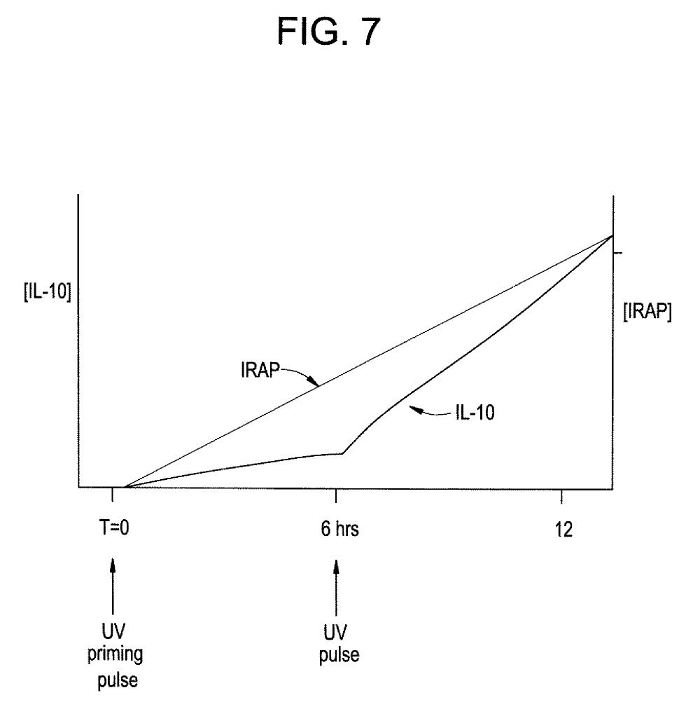

Now referring to FIG. 7, there is provided a graph of the relative concentrations of IL-10 and IRAP produced in accordance with a preferred embodiment of the present invention.

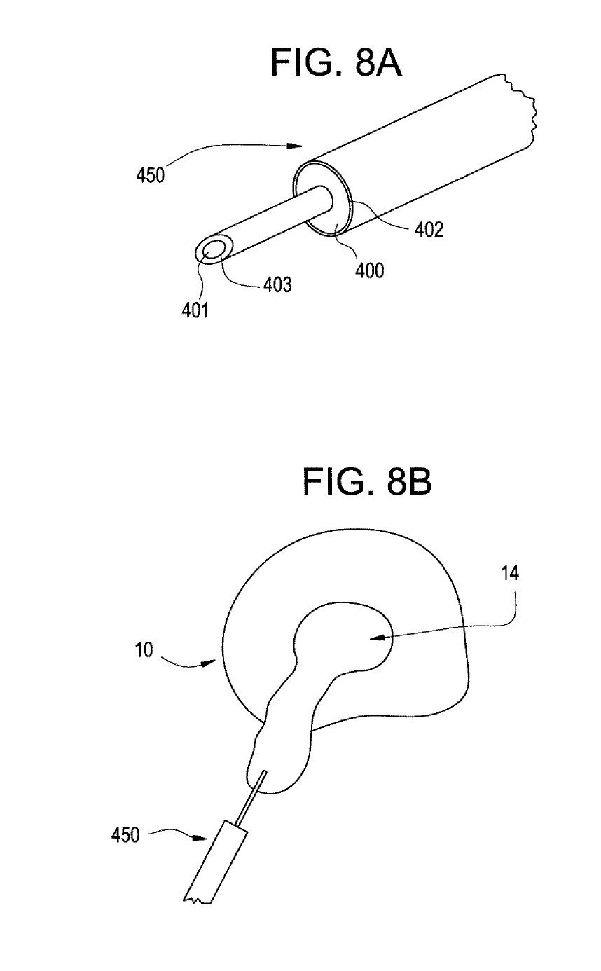

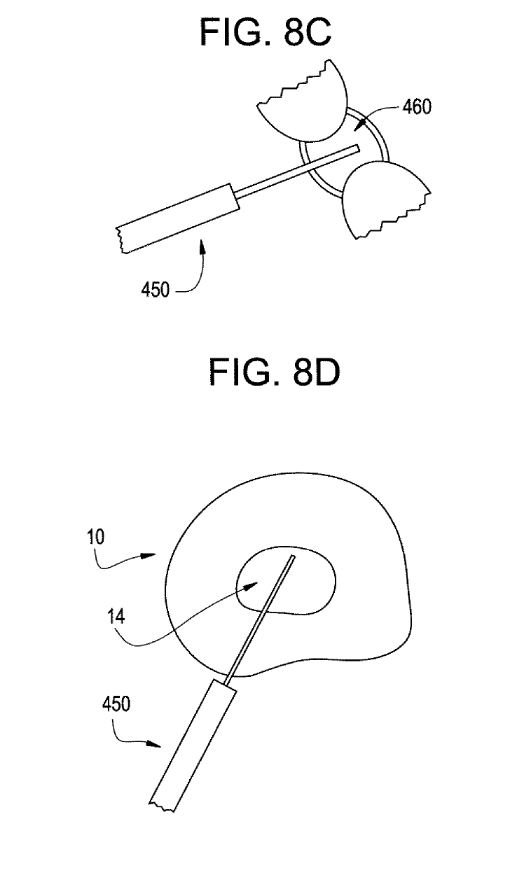

Now referring to FIG. 8a, there is provided a portion of a probe 450 of the present invention, comprising:
a) fiber optic cable 401, and
b) an inducing coating 403 (such as IgA or IgG) upon the fiber optic cable 401.

In some embodiments, cladding 400 is also coated with the inducer coating 402.

Now referring to FIG. 8b, there is provided the probe 450 of FIG. 8a inserted into the extruded portion of a nucleus pulposus 14 of disc 10. Now referring to FIG. 8c, there is provided the probe 450 of FIG. 8a inserted into the synovium 460 of an inflamed facet joint. Now referring to FIG. 8d, there is provided a probe of the present invention having a distal end portion inserted into the nucleus pulposus 14 of a herniated disc 10.

Because the probe of the present invention sits essentially passively during the refractory phase (in which IRAP is continually being produced), it has been observed that the probe need not be connected to the light source during this period.

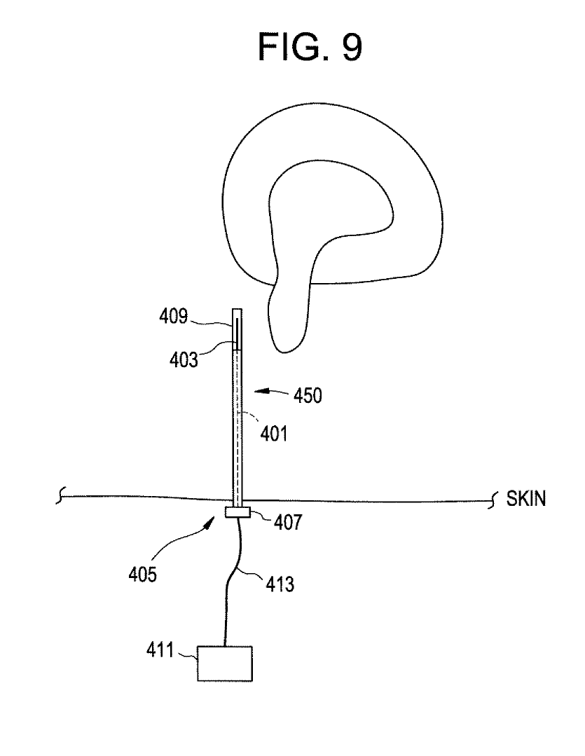

Therefore, in some embodiments, and now referring to FIG. 9, there is provided a probe 450, comprising:
a) a fiber optic cable 401 having a distal end portion 403 adapted to be inserted into a joint space and a proximal end portion 405 having a light port 407,
b) an inducer coating 409 provided upon a portion of the outer surface of the probe 450, and
c) a UVB source 411 having a distal end 413 adapted for reception in the light port 407.

In this embodiment, the clinician may first activate the UVB source to initiate the first production phase wherein UVB light activates Th2 lymphocytes and macrophages to emit IL-10; disengage the UV source from the light port to allow the patient freedom of movement for the refractory phase (a period of up to about 6 hours, during which IRAP is produced via the inducing coating), and then re-engage the light source to the light port in order to re-irradiate the primed macrophages and lymphocytes.

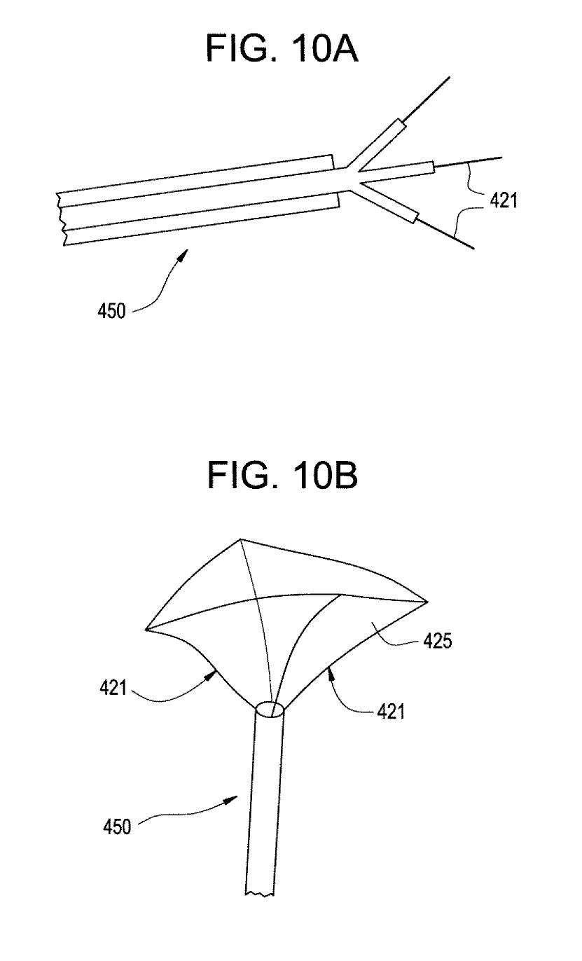

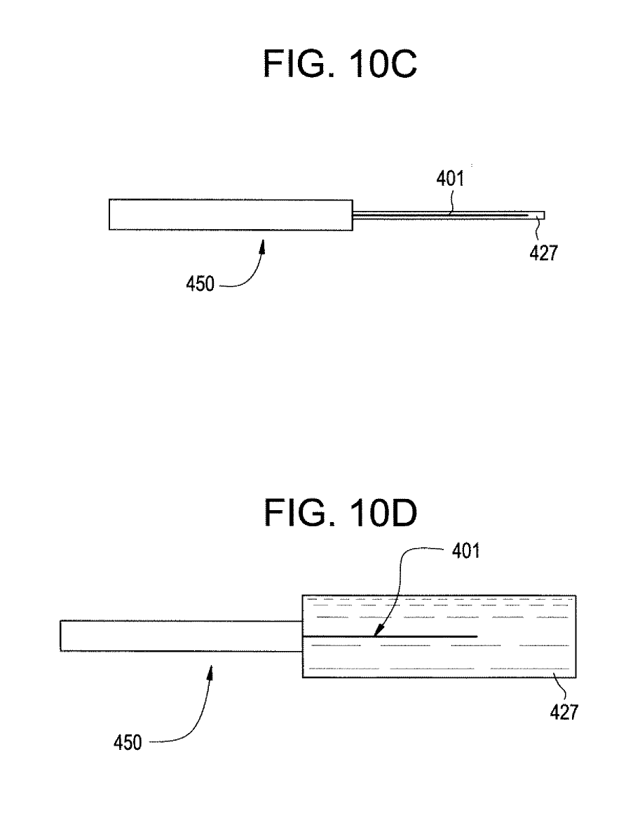

Because UV light does not significantly penetrate tissue, it is noted that both the UV light and the coating affect only cells in the immediate vicinity of the probe. In some embodiments, therefore, and now referring to FIG. 10a, there is provided a probe 450 having a plurality of distal end tynes 421. The plurality of distal end tynes 421 increases the surface area for UV light transmission and coating attachment, thereby increasing the effectiveness of the probe 450 in activating Th2 cells and macrophages.

FIG. 10b presents another means of increasing the area of activity for the probe 450 of the present invention. In this embodiment, a web 425 is formed between adjacent tynes 421. The web 425 may be coated with an inducer coating (such as IgG, IgA or IL-4), made of fiber optic material, or both.

FIG. 10c presents another means of increasing the area of activity for the probe 450 of the present invention. In this embodiment, the inducer material of the present invention is provided within a water swellable matrix 427 such as collagen that surround the fiber optic cable 401. Now referring to FIG. 10d, when inserted into an inflamed region, water invades the collagen matrix 427 and causes it to swell, thereby increasing the effective volume of treatable by the probe.

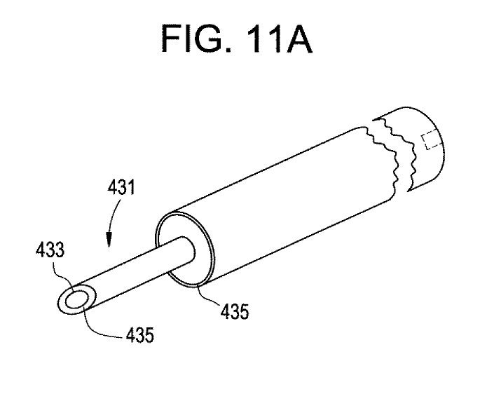

In some embodiments, now referring to FIG. 11a, the UV light source of the present invention may be completely eliminated so that the probe consists essentially of a distal tyne 431 having an outer surface 433 having inducer coating 435 (such as IgG, IgA or IL-4) thereon. In many embodiments, this probe must reside in the patient's joint for an extended period and so its proximal end is equipped with an attachment port for attachment and detachment to an instrument. Accordingly, this device allows mobility to the patient during the treatment period.

Therefore, in accordance with the present invention, there is provided a probe for treating spine-related pain, comprising:
a) a fiber optic cable, and
b) an inducing coating upon the fiber optic cable,
wherein the inducing coating is capable of inducing a macrophage to emit an anti-inflammatory cytokine.

We claim:

1. A method of treating DDD in an intervertebral disc, comprising the steps of:
a) providing a probe having a distal end portion having a UV light source,
b) positioning the UV light source within the disc, and
c) activating the light source to irradiate the disc in an amount of UV light sufficient
to activate macrophages therein.

2. A method of treating DDD in an intervertebral disc having a tear in an annulus fibrosus, comprising the steps of:
a) providing a probe having a distal end portion having a UV light source,
b) positioning the UV light source adjacent the tear, and
c) activating the light source to irradiate the disc in an amount of UV light sufficient to activate macrophages present in the vicinity.

3. A method of treating sciatica, comprising:
a) providing a probe having a distal end portion having a UV light source,
b) positioning the UV light source in the vicinity of the exuded nucleus pulposus, and
c) activating the light source to irradiate the exuded nucleus pulposus in an amount of UV light sufficient to activate macrophages present in the vicinity.

4. A method of treating a facet joint, comprising:
a) providing a probe having a distal end portion having a UV light source,
b) positioning the UV light source within a synovium of the facet joint, and
c) activating the light source to irradiate the synovium in an amount of UV light sufficient to activate macrophages and lympocytes present in the synovium.
